SEQUENCE LISTING (28 sequences)

SEQ ID NO: 1 - DNA, 907 bp, Mus musculus
tagcatctccgagagtttaaagctgaggaggcgggttcatgaaactgataaaaactgctcagaaggattatatcaaggaggcccattttgctgtcaaccatgccaacctggtaaaaaaaaagttgaggactgcaaaatgaatgggggtacaccaacctgtgccccatgcacagaagggaaggagtacatggacaagaaccattatgctgataaatgcagaagatgcacactctgcgatgaagagcatggtttagaagtggaaacaaactgcaccctgacccagaataccaagtgcaagtgcaaaccagacttctactgcgattctcctggctgtgaacactgtgttcgctgcgcctcgtgtgaacatggaacccttgagccatgcacagcaaccagcaatacaaactgcaggaaacaaagtcccagaaatcgcctatggttgttgaccatccttgttttgttaattccacttgtatttatatatcgaaagtaccggaaaagaaagtgctggaaaaggagacaggatgaccctgaatctagaacctccagtcgtgaaaccataccaatgaatgcctcaaatcttagcttgagtaaatacatcccgagaattgctgaagacatgacaatccaggaagctaaaaaatttgctcgagaaaataacatcaaggagggcaagatagatgagatcatgcatgacagcatccaagacacagctgagcagaaagtccagctgctcctgtgctggtaccaatctcatgggaagagtgatgcatatcaagatttaatcaagggtctcaaaaaagccgaatgtcgcagaaccttagataaatttcaggacatggtccagaaggaccttggaaaatcaaccccagacactggaaatgaaaatgaaggacaatgtctggag

SEQ ID NO: 2 - DNA, 21 bp, Artificial Sequence
  Chimeric oligonucleotide designed as RNA2-1. “nucleotides 1 to 19 are ribonucleotides-other nucleotides are deoxyribonucleotides”
gugcaagugcaaaccagactt

SEQ ID NO: 3 - DNA, 21 bp, Artificial Sequence
  Chimeric oligonucleotide designed as RNA2-2. “nucleotides 1 to 19 are ribonucleotides-other nucleotides are deoxyribonucleotides”
gucugguuugcacuugcactt

SEQ ID NO: 4 - DNA, 21 bp, Artificial Sequence
  Chimeric oligonucleotide designed as RNA3-1. “nucleotides 1 to 19 are ribonucleotides-other nucleotides are deoxyribonucleotides”
agccgaaugucgcagaacctt

SEQ ID NO: 5 - DNA, 21 bp, Artificial Sequence
  Chimeric oligonucleotide designed as RNA3-2. “nucleotides 1 to 19 are ribonucleotides-other nucleotides are deoxyribonucleotides”
gguucugcgacauucggcutt

SEQ ID NO: 6 - DNA, 21 bp, Artificial Sequence
  Chimeric oligonucleotide designed as RNA4-1. “nucleotides 1 to 19 are ribonucleotides-other nucleotides are deoxyribonucleotides”
aagccgaaugucgcagaactt

SEQ ID NO: 7 - DNA, 21 bp, Artificial Sequence
  Chimeric oligonucleotide designed as RNA4-2. “nucleotides 1 to 19 are ribonucleotides-other nucleotides are deoxyribonucleotides”
guucugcgacauucggcuutt

SEQ ID NO: 8 - DNA, 21 bp, Artificial Sequence
  Chimeric oligonucleotide designed as RNA5-1. “nucleotides 1 to 19 are ribonucleotides-other nucleotides are deoxyribonucleotides”
ggauuauaucaaggaggcctt

SEQ ID NO: 9 - DNA, 21 bp, Artificial Sequence
  Chimeric oligonucleotide designed as RNA5-2. “nucleotides 1 to 19 are ribonucleotides-other nucleotides are deoxyribonucleotides”
ggccuccuugauauaaucctt

SEQ ID NO: 10 - DNA, 21 bp, Artificial Sequence
  Chimeric oligonucleotide designed as RNA6-1. “nucleotides 1 to 19 are ribonucleotides-other nucleotides are deoxyribonucleotides”
aucgccuaugguuguugactt

SEQ ID NO: 11 - DNA, 21 bp, Artificial Sequence
  Chimeric oligonucleotide designed as RNA6-2. “nucleotides 1 to 19 are ribonucleotides-other nucleotides are deoxyribonucleotides”
gucaacaaccauaggcgautt

SEQ ID NO: 12 - DNA, 19 bp, Artificial Sequence
  Designed PCR primer to amplify a portion of mouse Fas gene.
cacagttaagagttcatac

SEQ ID NO: 13 - DNA, 19 bp, Artificial Sequence
  Designed PCR primer to amplify a portion of mouse Fas gene.
tggttgctgtgcatggctc

SEQ ID NO: 14 - DNA, 36 bp, Artificial Sequence
  Designed PCR primer rsGFP-1 to amplify a portion of rsGFP gene.
cagtcacgactctagaaaaggagaagaactcttcac

SEQ ID NO: 15 - DNA, 38 bp, Artificial Sequence
  Designed PCR primer rsGFP-2 to amplify a portion of rsGFP gene.
cagtcacgacgctagcagttgtacagttcatccatgcc

SEQ ID NO: 16 - DNA, 741 bp, Artificial Sequence
  rsGFP gene
cagtcacgactctagaaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaactgctagcgtcgtgactg

SEQ ID NO: 17 - DNA, 21 bp, Artificial Sequence
  Chimeric oligonucleotide designed as RNA7-1. “nucleotides 1 to 19 are ribonucleotides-other nucleotides are deoxyribonucleotides”
aagagagaccacauggucctt

SEQ ID NO: 18 - DNA, 21 bp, Artificial Sequence
  Chimeric oligonucleotide designed as RNA7-2. “nucleotides 1 to 19 are ribonucleotides-other nucleotides are deoxyribonucleotides”
ggaccauguggucucucuutt

SEQ ID NO: 19 - DNA, 21 bp, Artificial Sequence
  Chimeric oligonucleotide designed as RNA8-1. “nucleotides 1 to 19 are ribonucleotides-other nucleotides are deoxyribonucleotides”
gcguucaacuagcagaccatt

SEQ ID NO: 20 - DNA, 21 bp, Artificial Sequence
  Chimeric oligonucleotide designed as RNA8-2. “nucleotides 1 to 19 are ribonucleotides-other nucleotides are deoxyribonucleotides”
uggucugcuaguugaacgctt

SEQ ID NO: 21 - DNA, 21 bp, Artificial Sequence
  Chimeric oligonucleotide designed as RNA9-1. “nucleotides 1 to 19 are ribonucleotides-other nucleotides are deoxyribonucleotides”
agagagaccacaugguccutt

SEQ ID NO: 22 - DNA, 21 bp, Artificial Sequence
  Chimeric oligonucleotide designed as RNA9-2. “nucleotides 1 to 19 are ribonucleotides-other nucleotides are deoxyribonucleotides”
aggaccauguggucucucutt

SEQ ID NO: 23 - DNA, 21 bp, Artificial Sequence
  Chimeric oligonucleotide designed as RNA10-1. “nucleotides 1 to 19 are ribonucleotides-other nucleotides are deoxyribonucleotides”
guucucugucaguggagagtt

SEQ ID NO: 24 - DNA, 21 bp, Artificial Sequence
  Chimeric oligonucleotide designed as RNA10-2. “nucleotides 1 to 19 are ribonucleotides-other nucleotides are deoxyribonucleotides”
cucuccacugacagagaactt

SEQ ID NO: 25 - DNA, 20 bp, Artificial Sequence
  Designed PCR primer GFP-B-F to amplify a portion of rsGFP gene.
gccacaacattgaagatgga

SEQ ID NO: 26 - DNA, 20 bp, Artificial Sequence
  Designed PCR primer GFP-B-R to amplify a portion of rsGFP gene.
gaaagggcagattgtgtgga

SEQ ID NO: 27 - DNA, 20 bp, Artificial Sequence
  Designed PCR primer Neo-F to amplify a portion of neomycin resistant gene.
atagcgttggctacccgtga

SEQ ID NO: 28 - DNA, 20 bp, Artificial Sequence
  Designed PCR primer Neo-R to amplify a portion of neomycin resistant gene.
gaaggcgatagaaggcgatg

(19) United States
(12) Patent Application Publication
(10) Pub. No.: US 2007/0077563 A1
(43) Pub. Date: Apr. 5, 2007

(54) Method of searching for functional nucleotide molecule

(75) Inventors: Takashi Ueno, Shiga (JP); Masashige Tanabe, Shiga (JP); Risa Sumioka, Shiga (JP); Eiji Kobayashi, Shiga (JP); Nobuto Koyama, Shiga (JP); Hiroaki Sagawa, Shiga (JP); Junichi Mineno, Shiga (JP); Ikunoshin Kato, Shiga (JP)

Correspondence Address: BROWDY AND NEIMARK, P.L.L.C.;624 NINTH STREET, NW, SUITE 300, WASHINGTON, DC, 20001-5303, US

(73) Assignee: TAKARA BIO INC., Otsu-shi (JP)

(21) Appl. No.: 10/569,814

(22) PCT Filed: Aug. 25, 2004
(86) PCT No.: PCT/JP04/12172

§ 371 Date: Feb. 28, 2006

(30) Foreign Application Priority Data
Aug. 29, 2003 (JP) 2003307624

(51) Int. Cl. C12Q 1/68; C07H 21/02

(52) U.S. Cl. 435/6; 536/23.1

(57) ABSTRACT

A universal method of searching for a nucleic acid capable of effectively inhibiting gene expression; a nucleic acid construct to be used in this method; a vector; and a kit for the above method. This method is characterized in that a nucleic acid construct, which is a nucleic acid construct having a promoter sequence, at least two gene sequences and a poly A signal sequence and in which the above-described at least two gene sequences are transcribed as a single molecule RNA and at least one gene sequence is in the translatable state while at least one gene sequence is encoded in the substantially untranslatable state, is constructed.

### TECHNICAL FIELD

The present invention relates to a universal method of screening for a nucleic acid that is capable of effectively suppressing gene expression. The present invention further relates to a nucleic acid construct and a vector used for the method, as well as a kit for the method.

### BACKGROUND ART

Expression levels of proteins are controlled in various ways. A cell always maintains the desired expression levels, for example, by controlling transcription from genes into mRNAs using transcription factors, translation from mRNAs to amino acid sequences, and stabilities of mRNAs against degradation by nucleases.

Methods for artificially suppressing expression of a protein at mRNA level include suppression using an antisense RNA for an mRNA, degradation of an mRNA using a ribozyme, and RNA interference (RNAi). It is known that the suppressive effect of such a method may remarkably vary depending on the region in a nucleic acid sequence encoding the protein or the surrounding nucleic acid sequence to be selected as a target (e.g., Non-patent Document 1). According to a usual method of determining an expression level of a protein, a fusion protein of the target protein and a reporter protein is synthesized, and the expression level of the protein is estimated using the reporter protein as an index. In such a method, the reporter protein is usually fused downstream of the target protein in order to confirm that the entire fusion protein of the target protein and the reporter protein is translated. Thus, a DNA in which a reporter gene in a translatable state is linked to an adjacent nucleic acid sequence encoding a target protein in a nontranslatable state is not known.

For example, Nilsen, T. W. et al. screened for a ribozyme or the like that is capable of effectively suppressing expression of a target nucleic acid sequence by constructing a DNA for expressing a fusion protein of a target protein and a reporter protein fused downstream of the target protein, and examining the expression of the reporter protein (e.g., Patent Document 1). However, it is not easy to express a functional fusion protein. In not a few cases, functions of proteins are lost when they are fused. Thus, if the reporter protein is not detected, one needs to further verify in detail if it is due to destabilization of the mRNA caused by degradation, change in conformation of the mRNA, inhibition of the translation machinery, or loss of the function as a reporter protein. A method of screening for a molecule that acts on a sequence derived from a target gene without generating such a fusion protein or a chimeric protein is not known.

Thus, a method of screening for a functional nucleotide molecule that alters expression by altering stability of an mRNA, which can be universally applied to many nucleic acid sequences, has been desired.

Patent Document 1: United States Patent Publication No. 2002/0002278

Non-patent Document 1: Nature Medicine, Vol. 9, No. 3, pp. 347 (2003)

### DISCLOSURE OF INVENTION

Problems to be Solved by the Invention

The main object of the present invention is to provide a method of screening for a functional nucleotide molecule that alters expression of a target gene, which can be universally applied to many nucleic acid sequences.

Means to Solve the Problems

The present inventors have intensively conducted studies and searches in view of the above-mentioned situations. As a result, the present inventors have found the following. A nucleic acid construct having a promoter sequence, at least two gene sequences and a poly A signal sequence is constructed. The at least two gene sequences are transcribed in a single RNA molecule. At least one of the gene sequences is in a translatable state, and at least one of the gene sequences is encoded in a substantially nontranslatable state. Thereby, it is possible to screen for a molecule that can alter expression of the protein encoded by the nontranslatable gene sequence by altering stability of mRNA, and the method can be universally applied to many nucleic acid sequences. The present inventors have further found that the above-mentioned nucleic acid construct can be universally applied to many nucleic acid sequences. Thus, the present invention has been completed.

The present invention is outlined as follows. The first aspect of the present invention relates to a nucleic acid construct having a promoter sequence, at least one protein-encoding nucleotide sequence linked to the promoter sequence in a translatable state, and a poly A signal sequence, wherein

the nucleic acid construct further contains, between the promoter sequence and the poly A signal sequence, a nontranslatable nucleotide sequence that is different from the protein-encoding nucleotide sequence,

the protein-encoding nucleotide sequence linked to the promoter sequence in a translatable state and the nontranslatable nucleotide sequence that is different from the protein-encoding nucleotide sequence are linked together so that they are transcribed from the nucleic acid construct in a single RNA molecule, and

the nontranslatable nucleotide sequence is selected from the group consisting of:

(A) a nucleotide sequence that encodes a protein or a part of the protein; and

(B) a nucleotide sequence of an untranslated region that is naturally located on the 5′ or 3′ side of a nucleotide sequence that encodes a protein.

In the nucleic acid construct according to the first aspect, the nontranslatable nucleotide sequence may be located downstream of the protein-encoding nucleotide sequence linked to the promoter sequence in a translatable state. The nontranslatable nucleotide sequence may be located upstream of the protein-encoding nucleotide sequence linked to the promoter sequence in a translatable state. Furthermore, the protein-encoding nucleotide sequence linked to the promoter sequence in a translatable state in the nucleic acid construct may encode a reporter protein.

The second aspect of the present invention relates to a vector containing the nucleic acid construct of the first aspect.

The third aspect of the present invention relates to an RNA containing at least one protein-encoding nucleotide sequence in a translatable state and a nontranslatable nucleotide sequence that is different from the protein-encoding nucleotide sequence, wherein the nontranslatable nucleotide sequence is selected from the group consisting of:

(A) a nucleotide sequence that encodes a protein that is different from the protein-encoding nucleotide sequence in a translatable state, or a part of the protein; and

(B) a nucleotide sequence of an untranslated region that is naturally located on the 5′ or 3′ side of a nucleotide sequence that encodes a protein that is different from the protein-encoding nucleotide sequence in a translatable state.

The fourth aspect of the present invention relates to a method of detecting an activity of altering expression of a target gene by a functional nucleotide molecule, the method comprising the steps of:

(A) transcribing an RNA from the nucleic acid construct of the first aspect or the vector of the second aspect which has, as a nontranslatable nucleotide sequence, a nucleotide sequence selected from the group consisting of a nucleotide sequence that encodes a protein in a target gene, a part of the nucleotide sequence, and an untranslated region that is located on the 5′ or 3′ side of the nucleotide sequence that encodes the protein in the target gene;

(B) contacting a nucleotide molecule with the RNA transcribed in step (A);

(C) detecting the RNA in step (B) or a translation product translated from the RNA; and

(D) detecting an activity of altering expression of the target gene by a functional nucleotide molecule based on the amount of the RNA or the translation product translated from the RNA detected in step (C).

The fifth aspect of the present invention relates to a method of detecting an activity of altering expression of a target gene by a functional nucleotide molecule, the method comprising the steps of:

(A) contacting a nucleotide molecule with the RNA of the third aspect which has, as a nontranslatable nucleotide sequence, a nucleotide sequence selected from the group consisting of a nucleotide sequence that encodes a protein in a target gene, a part of the nucleotide sequence, and an untranslated region that is located on the 5′ or 3′ side of the nucleotide sequence that encodes the protein in the target gene;

(B) detecting the RNA in step (A) or a translation product translated from the RNA; and

(C) detecting an activity of altering expression of the target gene by a functional nucleotide molecule based on the amount of the RNA or the translation product translated from the RNA detected in step (B).

A method of screening for a functional nucleotide molecule that alters expression of a target gene may comprise detecting an activity of altering expression of a target gene by a functional nucleotide molecule according to the fourth or fifth aspect. In the method of detecting an activity of altering expression of a target gene by a functional nucleotide molecule according to the fourth or fifth aspect, the nucleotide molecule may be contacted with the RNA in a cell or in a cell-free protein synthesis system.

The sixth aspect of the present invention relates to a method of screening for a gene whose expression is altered by a nucleotide molecule, the method comprising the steps of:

(A) transcribing an RNA from the nucleic acid construct of the first aspect or the vector of the second aspect which has, as a nontranslatable nucleotide sequence, a nucleotide sequence selected from the group consisting of a nucleotide sequence that encodes a protein in an arbitrary gene, a part of the nucleotide sequence, and an untranslated region that is located on the 5′ or 3′ side of the nucleotide sequence that encodes the protein;

(B) contacting a nucleotide molecule with the RNA transcribed in step (A);

(C) detecting the RNA in step (B) or a translation product translated from the RNA; and

(D) identifying a gene whose expression is altered by the nucleotide molecule based on the amount of the RNA or the translation product translated from the RNA detected in step (C).

The seventh aspect of the present invention relates to a method of screening for a gene whose expression is altered by a nucleotide molecule, the method comprising the steps of:

(A) contacting a nucleotide molecule with the RNA of the third aspect which has, as a nontranslatable nucleotide sequence, a nucleotide sequence selected from the group consisting of a nucleotide sequence that encodes a protein in an arbitrary gene, a part of the nucleotide sequence, and an untranslated region that is located on the 5′ or 3′ side of the nucleotide sequence that encodes the protein;

(B) detecting the RNA in step (A) or a translation product translated from the RNA; and

(C) identifying a functional nucleotide molecule that alters expression of a target gene based on the amount of the RNA or the translation product translated from the RNA detected in step (B).

In the method of screening for a gene whose expression is altered by a nucleotide molecule according to the sixth or seventh aspect, the nucleotide molecule may be contacted with the RNA in a cell or in a cell-free protein synthesis system.

### EFFECTS OF THE INVENTION

The present invention provides a method of screening for a nucleotide molecule that suppresses expression of a target gene by destabilizing an RNA, which can be universally applied to many target genes.

### BRIEF DESCRIPTION OF DRAWINGS

FIG. 1 illustrates results of screening according to the method of screening a functional nucleotide molecule of the present invention.

FIG. 2 illustrates results of screening according to the method of screening a functional nucleotide molecule of the present invention.

FIG. 3 illustrates results of screening according to the method of screening a functional nucleotide molecule of the present invention.

FIG. 4 illustrates results of screening according to the method of screening a functional nucleotide molecule of the present invention.

### BEST MODE FOR CARRYING OUT THE INVENTION

Hereinafter, the present invention will be described in detail.

According to the present invention, a nucleic acid construct is a construct composed of a DNA and/or an RNA. The nucleic acid construct may be composed of, or may contain as a part thereof, an analog or a modified compound of a DNA or an RNA.

As used herein, a target gene refers to a nucleic acid sequence that encodes a target protein whose expression is desired to be altered and/or a nucleic acid sequence preceding or following it, which can be transcribed from the nucleic acid construct or the vector of the present invention. It may also be referred to as a nucleic acid sequence of interest or a target nucleic acid sequence herein. The target gene may be a nucleotide sequence that encodes an entire target protein or a part thereof, or a 5′UTR (untranslated region) or a 3′UTR preceding or following it. Although it is not intended to limit the present invention, for example, it is a functional nucleotide molecule such as a nucleotide sequence encoding a protein of interest whose expression is desired to be suppressed by an RNAi activity or a sequence preceding or following it (siRNA), and is a nucleic acid sequence to be subjected to sequence-specific mRNA degradation mediated by a nucleotide molecule. Also, a sequence corresponding to an exon in a gene, or a sequence modified not to contain a sequence that functions as an initiation codon can be preferably used. The target gene may be a sequence derived from a eukaryote, a sequence derived from a virus or a sequence derived from a prokaryote. A target gene derived from a virus is useful for screening for a functional nucleotide molecule involved in degradation of the genome of the virus, or suppression of replication or proliferation of the virus.

As used herein, a coding region means a region in a gene that consists of genetic codes directly defining an amino acid sequence of a protein.

As used herein, destabilization of an mRNA means increase in an mRNA degradation reaction. The amount of accumulated mRNA depends on two reactions, i.e., synthesis and degradation. An mRNA is said to be destabilized when the balance between the synthesis reaction and the degradation reaction is inclined to the degradation reaction. There is no specific limitation concerning the degradation of an mRNA as long as it is degradation of an mRNA that contains a nucleic acid sequence derived from a target gene. It may be endo-type or exo-type degradation.

As used herein, a nucleotide molecule refers to a phosphoester compound of nucleoside. The nucleotide molecule may be composed of ribonucleotide or deoxyribonucleotide, or it may be a chimeric molecule composed of them. The nucleotide molecules also include an analog or a modified nucleotide. It may form a complex with a protein, a sugar or the like.

As used herein, a functional nucleotide molecule refers to a nucleotide molecule that alters expression of a protein. Functional nucleotide molecules include a molecule that inhibits expression of a target protein, a molecule that acts on a nontranslatable nucleotide sequence in the RNA of the present invention in a sequence-specific manner to finally destabilize the whole RNA, and a molecule that degrades a nontranslatable nucleotide sequence in an RNA in a sequence-specific manner to finally destabilize the whole RNA. The functional nucleotide molecule may form a complex with a protein, a sugar or the like.

As used herein, a nucleotide sequence in a translatable state is a nucleotide sequence placed so that a protein is synthesized under appropriate conditions or environments. Although many factors are cooperatively involved in translation, the translatable state according to the present invention means that a nucleotide sequence contains minimal factors necessary for translation such as a translation initiation signal (a translation activator), a translation initiation codon and a translation termination codon.

According to the present invention, a nontranslatable nucleotide sequence means that a nucleotide sequence is in a form designed theoretically not to be translated or translated at a negligible trace level, in other words, that a nucleotide sequence is not translated substantially. Translation at a trace level, if observed, is substantially equivalent to lack of translation if the feature of the present invention that the influence due to formation of a fusion protein can be eliminated is not spoiled at that level. That is, an amino acid sequence encoded by a nontranslatable nucleotide sequence is not translated as a fusion protein with an amino acid sequence encoded by a nucleotide sequence in a translatable state. A nontranslatable nucleotide sequence is a sequence that is not translated due to lack of a minimal factor necessary for translation.

As used herein, upstream and downstream represent positions relative to the direction of transcription of an RNA from a promoter in the nucleic acid construct of the present invention. In the nucleic acid construct of the present invention, positions proximal to a promoter sequence are upstream, whereas positions proximal to a poly A signal sequence are downstream.

(1) The Nucleic Acid Construct of the Present Invention

The nucleic acid construct of the present invention is a nucleic acid construct having a promoter sequence, at least one protein-encoding nucleotide sequence linked to the promoter sequence in a translatable state, and a poly A signal sequence, wherein

the nucleic acid construct further contains, between the promoter sequence and the poly A signal sequence, a nontranslatable nucleotide sequence that is different from the protein-encoding nucleotide sequence,

the protein-encoding nucleotide sequence linked to the promoter sequence in a translatable state and the nontranslatable nucleotide sequence that is different from the protein-encoding nucleotide sequence are linked together so that they are transcribed from the nucleic acid construct in a single RNA molecule, and

the nontranslatable nucleotide sequence is selected from the group consisting of:

(A) a nucleotide sequence that encodes a protein or a part of the protein; and

(B) a nucleotide sequence of an untranslated region that is naturally located on the 5′ or 3′ side of a nucleotide sequence that encodes a protein.

An exemplary embodiment of the nucleic acid construct of the present invention is a nucleic acid construct having a promoter sequence, a poly A signal sequence, and a DNA sequence inserted between the promoter sequence and the poly A signal sequence which is transcribed in a single RNA molecule by the action of the promoter, wherein the DNA sequence is selected from the group consisting of:

(A) a DNA sequence having a nontranslatable nucleotide sequence linked to a reporter gene sequence, wherein an RNA transcribed from the DNA encodes a product of the reporter gene in a translatable state and the nucleotide sequence in a nontranslatable state; and

(B) a DNA sequence having a reporter gene sequence and a restriction enzyme recognition/cleavage site adjacent thereto, wherein if a nucleotide sequence is inserted at the restriction enzyme recognition/cleavage site, an RNA transcribed from the DNA encodes a product of the reporter gene in a translatable state and the nucleotide sequence in a nontranslatable state.

Although it is not intended to limit the present invention, the nontranslatable nucleotide sequence is usually derived from a gene different from the nucleotide sequence in a translatable state. Although it is not intended to limit the present invention, for example, the nucleic acid construct of the present invention may be a nucleic acid construct in which a promoter sequence, a reporter gene sequence, a sequence that functions as a termination codon, a nontranslatable nucleotide sequence and a poly A signal sequence are arranged in this order. Alternatively, it may be a nucleic acid construct in which a promoter sequence, a nontranslatable nucleotide sequence, a sequence that functions as a initiation codon, a reporter gene sequence and a poly A signal sequence are arranged in this order.

A nontranslatable nucleotide sequence (e.g., a sequence derived from a target gene) in the nucleic acid construct of the present invention exists as a UTR in an RNA transcribed from the nucleic acid construct and is not translated. Thus, a fusion protein is not generated, and a protein retaining its function is expressed from a protein-encoding encoding nucleotide sequence (e.g., a gene sequence encoding a reporter protein) linked in a translatable state. In the presence of a nucleotide molecule that degrades a region in the RNA that encodes the nontranslatable nucleotide sequence (e.g., a ribozyme or an siRNA), the whole RNA is destabilized, resulting in decreased expression of the protein encoded by the nucleotide sequence linked in a translatable state. Then, it is possible to universally and readily screen for a molecule that is capable of effectively suppressing expression of a nontranslatable nucleotide sequence by tracing change in an RNA transcribed from the nucleic acid construct of the present invention or a translation product from the RNA.

A nontranslatable nucleotide sequence may be located upstream or downstream of a protein-encoding nucleotide sequence linked in a translatable state as long as it is a sequence that is not translated substantially. Furthermore, the nontranslatable nucleotide sequence may be derived from a gene different from a protein-encoding nucleotide sequence linked in a translatable state.

If a nontranslatable nucleotide sequence is located upstream of a protein-encoding nucleotide sequence linked in a translatable state, there is no specific limitation concerning the nucleic acid construct of the present invention as long as the nontranslatable nucleotide sequence is placed so that it is not translated. For example, a certain region may be selected not to contain a translation initiation codon (e.g., ATG) to make the sequence in a nontranslatable state. If there is a sequence that has a possibility of functioning as an initiation codon, the codon may be modified by substitution, addition, deletion or insertion of a nucleotide to make the sequence nontranslatable.

If a nontranslatable nucleotide sequence is located downstream of a protein-encoding nucleotide sequence linked in a translatable state, there is no specific limitation concerning the nucleic acid construct of the present invention as long as the nontranslatable nucleotide sequence is placed so that it is not translated. For example, a sequence that functions as a termination codon (TAA, TAG, TGA) may be placed between a protein-encoding nucleotide sequence linked in a translatable state and the nontranslatable nucleotide sequence. In this case, a reading frame of three consecutive nucleotides (codon) starting from an initiation codon in the protein-encoding nucleotide sequence linked in a translatable state defines the termination codon, the termination codon terminates the translation of the RNA at the end of the protein-encoding nucleotide sequence linked in a translatable state, and the downstream nontranslatable nucleotide sequence is not translated. Thus, it is expected that a fusion protein of a protein encoded by a protein-encoding nucleotide sequence linked in a translatable state and a protein encoded by a nontranslatable nucleotide sequence is not generated, and the translation product of the protein-encoding nucleotide sequence linked in a translatable state is expressed while reflecting the stability of the mRNA.

The nucleic acid construct of the present invention can be used for a method of detecting alteration in gene expression by a functional nucleotide molecule.

Any promoter sequence may be contained in the nucleic acid construct of the present invention as long as it is a sequence that has an activity involved in an RNA transcription initiation reaction in a eukaryotic cell or an environment similar thereto (e.g., a cell-free protein expression system). A promoter sequence derived from a eukaryote (e.g., β-actin promoter, U6 promoter) or a promoter sequence derived from a virus that has a promoter activity in a eukaryotic cell (e.g., CMV (cytomegalovirus) promoter) may be used. Furthermore, it is possible to select a promoter suitable for the environment in which a transcription reaction is conducted (a biological individual, a cultured cell, a cell-free protein synthesis system, etc.). For example, in case of a cell-free protein synthesis system, one corresponding to the RNA polymerase to be used may be selected. Besides the above-mentioned promoter sequence derived from a eukaryote or a virus, a promoter sequence derived from a phage such as T7 phage may be selected. Although it is not intended to limit the present invention, in case of screening for a functional nucleotide molecule that suppresses expression of a protein, a constitutively and strongly expressing promoter can be preferably used such that the degree of suppression can be clearly judged.

The protein-encoding nucleotide sequence linked in a translatable state contained in the nucleic acid construct of the present invention is exemplified by a reporter gene sequence. There is no specific limitation concerning the reporter gene sequence as long as it is a nucleic acid sequence of a gene encoding an arbitrary protein that can be detected directly and/or indirectly. There is no specific limitation concerning the protein encoded by the reporter gene (reporter protein). Examples thereof include enzymes that produce substances that can be specifically detected (β-galactosidase, luciferase, alkaline phosphatase, etc.) and proteins that can be directly detected. Only a part of a reporter gene may be selected and used as long as the useful feature is retained. Plural reporter genes may be used in combination. Examples of methods in which proteins are directly detected include a method in which detection is carried out by using a specific antibody that recognizes a reporter protein, a method in which fluorescence from a reporter protein that emits a fluorescent signal (e.g., green fluorescence protein (GFP)) is detected, and use of a selectable marker protein such as one that confers a drug-resistant property. It is possible to sort cells using such a reporter protein. For example, a reporter protein that emits a fluorescent signal is useful because a cell expressing the protein can be selected using the FACS (fluorescence activated cell sorting) method.

The nontranslatable nucleotide sequence contained in the nucleic acid construct of the present invention may be of any region of a target gene of interest (or whose expression is desired to be altered). A coding region for a target protein, a part of the coding region, a 3′UTR or a 5′UTR can be selected as a nontranslatable nucleotide sequence. Furthermore, sequences derived from plural genes may be arranged in the nontranslatable nucleotide sequence. In addition, a nucleic acid (population) prepared from a genomic library or a cDNA library derived from an organism of interest, a cDNA library representing expression in a specific organ or at a specific stage or the like may be used as a nontranslatable nucleotide sequence.

The nucleic acid construct of the present invention is transcribed in a single RNA molecule in a host cell into which it is transferred or in a transcription reaction mixture. The RNA can be destabilized with a functional nucleotide molecule. The functional nucleotide molecule is exemplified by a molecule that recognizes and acts on a nucleotide sequence of a region of a nontranslatable nucleotide sequence in an RNA to consequently destabilize the whole RNA. For example, the method of the present invention can be used to examine the activity of the following: a dsRNA, an siRNA, an shRNA and a nuclease complex (RNA-induced silencing complex (RISC)) in an RNAi mechanism, an stRNA (small temporal RNA) which is considered to be involved in a developmental stage of an organism, an miRNA (micro RNA) which is considered to be involved in wide variety of biological events, a protein complex miRNP which contains an miRNA, a ribozyme, a maxizyme, a hammerhead ribozyme, an antisense RNA, an EGS (external guide sequence) and a protein complex containing such a nucleotide molecule.

A protein (e.g., a reporter protein) encoded by a protein-encoding nucleotide sequence linked in a translatable state is stably expressed from an RNA transcribed from the nucleic acid construct of the present invention, without forming a fusion protein, as an individual protein, in an almost natural state. For example, if expression of a reporter protein is suppressed, there is a possibility that this phenomenon is caused by alteration in the inherent function of the reporter protein as a result of formation of a fusion protein of the protein encoded by the target gene and the reporter protein. It is possible to eliminate such a possibility according to the method of detecting gene expression of the present invention. It is also possible to eliminate a possibility that the phenomenon is caused by alteration in the inherent function of the reporter protein as a result of action of a functional nucleotide molecule on a nontranslatable nucleotide sequence to inhibit the translation mechanism.

In addition, since it is not necessary to translate a nontranslatable nucleotide sequence into a protein, it is not necessary to fit the reading frame (codon; a chain of three nucleotides corresponding to each amino acid) of the nontranslatable nucleotide sequence. Thus, a nontranslatable nucleotide sequence can be readily linked to a protein-encoding nucleotide sequence linked in a translatable state in the nucleic acid construct of the present invention because it is not necessary to select an appropriate restriction enzyme recognition site or to fit the reading frame, for example, by insertion or deletion of a nucleotide.

The nucleic acid constructs of the present invention include a nucleic acid construct having a protein-encoding nucleotide sequence linked in a translatable state and a restriction enzyme recognition/cleavage site adjacent thereto, wherein if a nontranslatable nucleotide sequence is inserted into the restriction enzyme recognition/cleavage site, an RNA transcribed from the nucleic acid construct encodes the protein-encoding nucleotide sequence linked in a translatable state so that it can be translated, and encodes the nontranslatable nucleotide sequence so that it is not translated substantially. Such a nucleic acid construct is useful because a target gene sequence can be inserted upon use depending on the object at will. Any restriction enzyme recognition/cleavage site may be used as along as it is a sequence convenient for insertion of a target gene sequence of interest. Although it is not intended to limit the present invention, for example, a sequence called a cloning site or a multiple cloning site in which one or plural restriction enzyme site(s) is (are) arranged may be used. A sequence widely and commonly used in a commercially available vector or linker can be preferably used.

The poly A signal sequence in the nucleic acid construct of the present invention is a sequence that causes a poly A addition reaction at a 3′ end of an mRNA. There is no specific limitation concerning the poly A signal sequence as long as it is a sequence that causes a poly A addition reaction. For example, the nucleotide sequence AAUAAA which is highly conserved among mRNAs from higher eukaryotes and is normally present 11 to 30 nucleotides upstream of a poly A addition site may be used.

A terminator sequence may be placed downstream of a poly A signal sequence in the nucleic acid construct of the present invention. Any terminator sequence may be used as long as it is a sequence that has a function of terminating transcription of an mRNA by an RNA polymerase. Although it is not intended to limit the present invention, an RNA polymerase terminates RNA synthesis at plural sites within a terminator sequence in a eukaryote.

A poly A signal sequence and a terminator sequence contained in the nucleic acid construct of the present invention can be appropriately selected depending on the environment in which an RNA transcription reaction is conducted. Many protein expression vectors adapted to hosts are commercially available. The nucleic acid construct of the present invention may be prepared on the basis of or combining such vectors. Although it is not intended to limit the present invention, for example, bovine-derived BGH poly A signal sequence and terminator sequence can be used.

(2) The Vector of the Present Invention

The vector of the present invention is a vector that contains the nucleic acid construct of the present invention. It may be any vector from which an RNA containing, in the same molecule, a protein-encoding nucleotide sequence linked in a translatable state (e.g., a reporter gene sequence), a nontranslatable nucleotide sequence (e.g., a target gene sequence) and a poly A signal sequence is transcribed in an appropriate host cell or a reaction mixture similar to the environment of the host cell. The vectors of the present invention include a vector used for constructing the vector of the present invention as long as it contains the nucleic acid construct of the present invention. The vector of the present invention may be a plasmid vector, a phage vector, an autonomously replicable vector, a virus vector, a vector integrated into host chromosome, or a vector for temporal or transient expression. It is preferable to select a vector for, or a host of, a compatible species depending on a promoter sequence, a gene sequence and a functional nucleotide molecule contained in the nucleic acid construct of the present invention. One can select one suitable for the object from many currently commercially available host-vector systems.

(3) The RNA of the Present Invention

The RNA of the present invention is an RNA containing at least one protein-encoding nucleotide sequence in a translatable state and a nontranslatable nucleotide sequence that is different from the protein-encoding nucleotide sequence, wherein the nontranslatable nucleotide sequence is selected from the group consisting of:

(A) a nucleotide sequence that encodes a protein that is different from the protein-encoding nucleotide sequence in a translatable state, or a part of the protein; and

(B) a nucleotide sequence of an untranslated region that is naturally located on the 5′ or 3′ side of a nucleotide sequence that encodes a protein that is different from the protein-encoding nucleotide sequence in a translatable state.

The RNA of the present invention can be prepared by in vivo or in vitro transcription of the nucleic acid construct of the present invention. Alternatively, it can be prepared by connecting RNAs as components constituting the RNA of the present invention.

The RNA of the present invention can be used for the method of screening for a functional nucleotide molecule that alters expression of a target gene or the method of screening for a gene whose expression is altered by a nucleotide molecule according to the present invention.

(4) The Method of Detecting an Activity of Altering Expression of a Target Gene by a Functional Nucleotide Molecule of the Present Invention

The method of detecting an activity of altering expression of a target gene by a functional nucleotide molecule of the present invention comprises the steps of:

(A) transcribing an RNA from the nucleic acid construct as described in (1) or the vector as described in (2) which has, as a nontranslatable nucleotide sequence, a nucleotide sequence selected from the group consisting of a nucleotide sequence that encodes a protein in a target gene, a part of the nucleotide sequence, and an untranslated region that is located on the 5′ or 3′ side of the nucleotide sequence that encodes the protein in the target gene;

(B) contacting a nucleotide molecule with the RNA transcribed in step (A);

(C) detecting the RNA in step (B) or a translation product translated from the RNA; and

(D) detecting an activity of altering expression of the target gene by a functional nucleotide molecule based on the amount of the RNA or the translation product translated from the RNA detected in step (C).

In one embodiment, the method of screening for a functional nucleotide molecule of the present invention comprises the steps of:

(A) preparing the nucleic acid construct or the vector of the present invention, that is, a nucleic acid construct having a promoter sequence, a poly A signal sequence, and a DNA sequence inserted between the promoter sequence and the poly A signal sequence which is transcribed in a single RNA molecule by the action of the promoter, or a vector containing the nucleic acid construct, wherein the DNA sequence is a DNA sequence having a nontranslatable nucleotide sequence linked to a reporter gene, wherein an RNA transcribed from the DNA encodes a product of the reporter gene in a translatable state and the nucleotide sequence in a nontranslatable state;

(B) transcribing an RNA from the nucleic acid construct or the vector in step (A);

(C) contacting a nucleotide molecule with the RNA in step (B); and

(D) detecting a region of the reporter gene in the RNA or a reporter protein.

An activity of altering expression of a target gene by a functional nucleotide molecule can be detected by: transcribing, using the nucleic acid construct of the present invention or a vector containing the nucleic acid construct, an RNA which contains a series of sequences including a protein-encoding nucleotide sequence linked in a translatable state, a nontranslatable nucleotide sequence and a poly A signal sequence, or preparing the RNA of the present invention; contacting a nucleotide molecule with the RNA; and detecting and comparing a part of the RNA corresponding to the protein-encoding nucleotide sequence linked in a translatable state or the translated protein. As a result of the above-mentioned detection, it is possible to judge that a nucleotide that results in alteration is a functional nucleotide. Furthermore, according to the present invention, it is possible to screen plural nucleotide molecules for a functional nucleotide molecule that alters expression of a target gene using the above-mentioned detection method. Transcription of an RNA, contact with a nucleotide molecule and expression of a protein may be carried out inside or outside a cell in parallel or successively. There is no specific limitation concerning the method of screening for a functional nucleotide molecule. For example, the nucleic acid construct of the present invention or the vector of the present invention and a functional nucleotide molecule are transferred into a host cell, and expression of a protein-encoding nucleotide sequence linked in a translatable state is examined.

The functional nucleotide molecule may be chemically synthesized on the basis of a target nucleic acid sequence or a part thereof, or produced by biosynthesis. Alternatively, it may be a product expressed in a host cell having a transferred vector designed so that it is produced in the host cell. Furthermore, plural functional nucleotide molecules such as plural nucleotides derived from a genomic library, a cDNA library or an organ/stage-specific cDNA library be used. One can screen for a functional nucleotide molecule that alters expression of a target gene by using such a library.

According to the method of the present invention, it is possible to directly detect an RNA or to detect a translation product from the RNA after contacting a nucleotide molecule with the RNA which has been transcribed from the nucleic acid construct or the vector of the present invention, or which is the RNA of the present invention. Any one of many methods for directly detecting an RNA which have been reported may be used as long as it enables qualitative or quantitative detection of an RNA. Examples thereof include, but are not limited to, the concentration measurement, the gel electrophoresis method, the PCR (Polymerase Chain Reaction) method, the real-time PT-PCR method, the TAS (Transcription-based Amplification System) method, the 3SR (Self-Sustained Sequence Replication), the NASBA (Nucleic Acid Sequence-Based Amplification) method, the Qβ replicase method and the TMA (Transcription Mediated Amplification) method.

A method of transferring the nucleic acid construct of the present invention, the vector of the present invention, a functional nucleotide molecule or a nucleic acid construct capable of producing a functional oligonucleotide molecule into a host cell may be selected such that it is suitable for the nucleic acid construct or the vector. Although it is not intended to limit the present invention, direct transfer using a physical or chemical means, or transfer by infection may be used.

(5) The Method of Screening for a Gene Whose Expression is Altered by a Nucleotide Molecule of the Present Invention

The method of screening for a gene whose expression is altered by a nucleotide molecule of the present invention comprises the steps of:

(A) transcribing an RNA from the nucleic acid construct of the first aspect or the vector of the second aspect which has, as a nontranslatable nucleotide sequence, a nucleotide sequence selected from the group consisting of a nucleotide sequence that encodes a protein in an arbitrary gene, a part of the nucleotide sequence, and an untranslated region that is located on the 5′ or 3′ side of the nucleotide sequence that encodes the protein;

(B) contacting a nucleotide molecule with the RNA transcribed in step (A);

(C) detecting the RNA in step (B) or a translation product translated from the RNA; and

(D) identifying a gene whose expression is altered by the nucleotide molecule based on the amount of the RNA or the translation product translated from the RNA detected in step (C).

According to the method of screening for a gene whose expression is altered by a nucleotide molecule of the present invention, it is possible to screen for a gene whose expression is altered by a functional nucleotide molecule, or to screen for an activity of such a functional nucleotide molecule by: producing plural target sequences from a genomic library, a cDNA library or an organ/stage-specific cDNA library as nontranslatable nucleotide sequences to prepare the nucleic acid constructs or the vectors of the present invention; transcribing plural RNAs containing the plural nontranslatable nucleotide sequences, or preparing the RNAs of the present invention; contacting a functional nucleotide molecule with the RNAs; and detecting a part of an RNA corresponding to a protein-encoding nucleotide sequence linked in a translatable state or a translated protein. A nucleic acid construct having a promoter sequence, a poly A signal sequence, and a DNA sequence inserted between the promoter sequence and the poly A signal sequence which is transcribed in a single RNA molecule by the action of the promoter can be preferably used for the method. The DNA sequence is a DNA sequence having a reporter gene and a restriction enzyme recognition/cleavage site adjacent thereto, wherein if a target gene sequence is inserted at the restriction enzyme recognition/cleavage site, an RNA transcribed from the DNA sequence encodes the reporter gene so that the product can be translated, and encodes the target gene sequence so that the product is not translated.

(6) The Kit of the Present Invention

The present invention provides kit used for the method of screening for a functional nucleotide molecule or the method of screening for a gene whose expression is altered by a nucleotide molecule according to the present invention as illustrated in (4) or (5) above. In one embodiment, the kits of the present invention include a kit that is in a packaged form and contains the nucleic acid construct of the present invention as illustrated in (1) above or a nucleic acid construct containing a cloning site in place of the nontranslatable gene sequence in the above-mentioned nucleic acid construct, into which a user can insert a sequence derived from a target gene of interest. The kit of the present invention may contain instructions.

“Instructions” are printed matters describing a method of using the kit, e.g., a method of preparing a reagent solution, recommended reaction conditions and the like. The instructions include an instruction manual in a form of a pamphlet or a leaflet, a label stuck to the kit, and description on the surface of the package containing the kit. The instructions also include information disclosed or provided through electronic media such as the Internet.

Furthermore, the kits of the present invention include a kit containing the vector as illustrated in (2) or the RNA as illustrated in (3).

### EXAMPLES

The following Examples illustrate the present invention in more detail, but are not to be construed to limit the scope thereof. Among the procedures described herein, basic procedures including preparation of plasmid DNAs and restriction enzyme digestion were carried out as described in Molecular Cloning: A Laboratory Manual, 2nd ed. Unless otherwise stated, Escherichia coli JM109 was used as a host for the construction of plasmids using Escherichia coli. Transformed Escherichia coli cells were cultured aerobically at 37° C. using LB medium (1% Tryptone, 0.5% yeast extract, 0.5% NaCl, pH 7.0) containing 100 μg/ml of ampicillin or LB-ampicillin plate prepared by adding agar at concentration of 1.5% to the above-mentioned medium and solidifying the resulting mixture. Cells were cultured using, as a medium, Dulbecco's modified Eagle's medium (Bio Whittaker) supplemented with 10% fetal calf serum (Bio Whittaker) in a cell culture dish (Iwaki Glass) at 37° C. in the presence of 5% CO₂ in a humidified culture incubator. Kits were used and various instruments were manipulated according to attached instructions.

### Example 1

### Construction of Target Plasmid Having Mouse Fas Gene Sequence

A plasmid pQBI25 (Wako Chemicals USA) has the CMV promoter, the rsGFP (red shifted green fluorescence protein) gene and the BGH poly A, and efficiently expresses rsGFP in cells. Sites for restriction enzymes BamHI and EcoRI exist between the rsGFP gene and the BGH poly A. A dsDNA (SEQ ID NO:1) having a sequence from 75 nucleotides downstream of the initiation codon to the termination codon of the mouse Fas gene (GenBank Accession: M83649) with cohesive ends for restriction enzymes BamHI and EcoRI added at the ends was inserted between the BamHI and EcoRI sites in pQBI25 to construct a target plasmid pTargetFas. The target plasmid transcribes an RNA having the rsGFP gene sequence and a partial sequence of the Fas gene in cells.

### Example 2

### Screening for siRNA that Effectively Suppresses Expression of Fas Gene

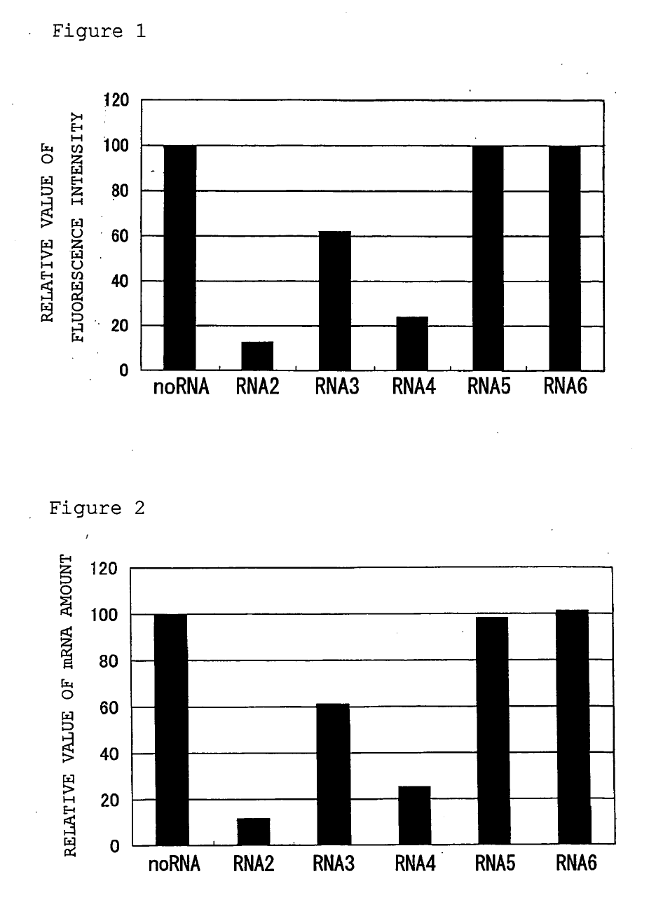

Five 21-bp dsRNAs each having a partial sequence of the mouse Fas gene were prepared. RNA2, RNA3, RNA4, RNA5 and RNA6 were prepared by annealing RNA2-1 (SEQ ID NO:2) to RNA2-2 (SEQ ID NO:3), RNA3-1 (SEQ ID NO:4) to RNA3-2 (SEQ ID NO:5), RNA4-1 (SEQ ID NO:6) to RNA4-2 (SEQ ID NO:7), RNA5-1 (SEQ ID NO:8) to RNA5-2 (SEQ ID NO:9), and RNA6-1 (SEQ ID NO:10) to RNA6-2 (SEQ ID NO:11), respectively. Each dsRNA was transferred into 293 cells (ATCC No. CRL-1573) along with pTargetFas. Gene transfer was carried out using Lipofectamine 2000 (Invitrogen) and Ribojuice (Takara Bio). The cells were cultured for two days and detached from the dish using trypsin, and fluorescence intensity was measured for the cells using MoFlo (Takara Bio). The results are shown in FIG. 1. FIG. 1 shows relative values defining the fluorescence intensity for control cells without the RNA transfer as 100. The weakest fluorescent intensity was observed for the cells into which RNA2 and pTargetFas were transferred. Thus, RNA2 was judged to be an siRNA that effectively suppresses expression of the Fas gene.

### Example 3

### Confirmation of Results in Example 2

The effectiveness of the siRNA obtained in Example 2 was confirmed as follows. Ribojuice (Takara Bio) was used to transfer RNA2, RNA3, RNA4, RNA5 or RNA6 into NIH3T3 cells (ATCC No. CRL-1658) which express Fas. After two days, RNAs were extracted from the cells, and Fas mRNAs were quantified using real-time RT-PCR. Cells without the RNA transfer were used as a control, and the data were corrected using a house-keeping gene GAPDH (glyceraldehyde-3-phosphate dehydrogenase). The real-time RT-PCR was carried out using Real Time RT-PCR Core Kit (Takara Bio) and Smart Cycler System (Takara Bio) as well as the oligo DNAs of SEQ ID NOS:12 and 13 as primers for Fas or the primers of Real Time RT-PCR Primer (Takara Bio) as primers for GAPDH. The results are shown in FIG. 2. FIG. 2 shows relative values defining the mRNA amount for control cells without the RNA transfer as 100. The decreases in Fas mRNA amounts were consistent with the decreases in rsGFP fluorescence intensities observed in Example 2. Thus, it was confirmed that this screening method is useful.

### Example 4

### Construction of Target Plasmid Having rsGFP (Red Shifted Green Fluorescence Protein) Gene Sequence

A DNA fragment (SEQ ID NO:16) that contains a region from the initiation codon to a portion around the termination codon in the rsGFP gene and has sites for restriction enzymes XbaI and NheI at the ends was prepared by a PCR using rsGFP-1 (SEQ ID NO:14) and rsGFP-2 (SEQ ID NO:15) as primers as well as pQBI25 as a template. The DNA fragment was treated with restriction enzymes XbaI and NheI to obtain an about 720-bp DNA fragment. The about 720-bp DNA fragment was inserted at a site for a restriction enzyme XbaI present between the firefly luciferase gene and the SV40 poly A in a plasmid pGL3control (Promega) to construct a target plasmid pGL3-3′ (GFP).

### Example 5

### Screening for siRNA that Effectively Suppresses Expression of rsGFP Gene

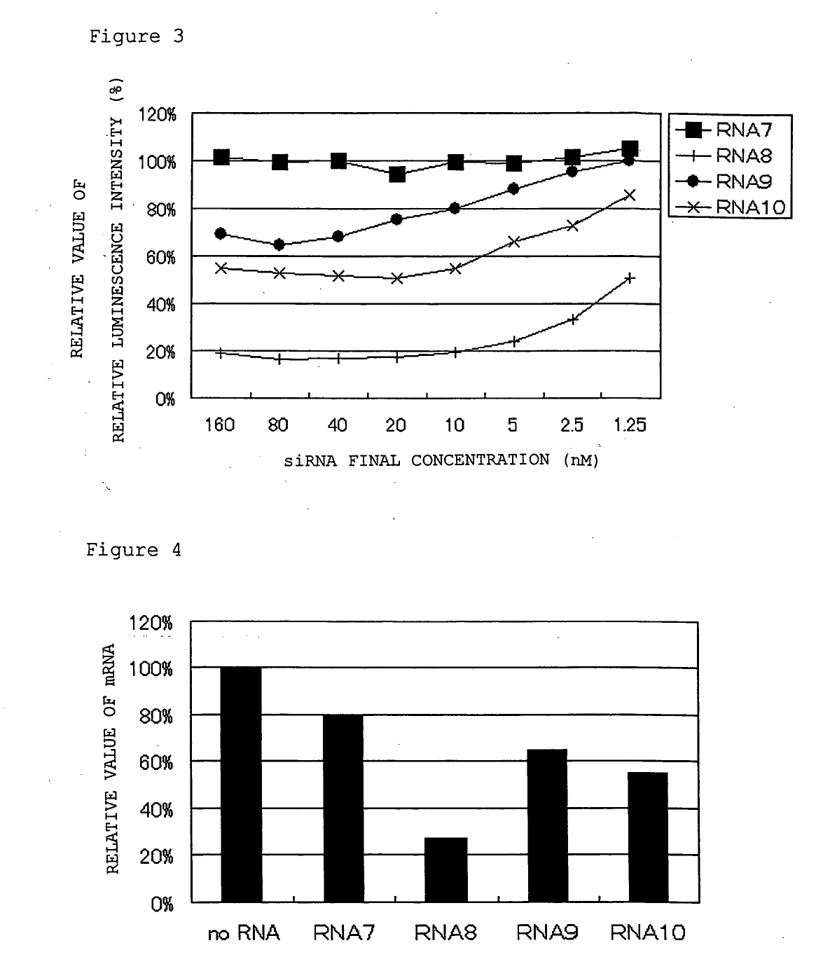

Five 21-bp dsRNAs each having a partial sequence of the rsGFP gene were prepared. RNA7, RNA8, RNA9 and RNA10 were prepared by annealing RNA7-1 (SEQ ID NO:17) to RNA7-2 (SEQ ID NO:18), RNA8-1 (SEQ ID NO:19) to RNA8-2 (SEQ ID NO:20), RNA9-1 (SEQ ID NO:21) to RNA9-2 (SEQ ID NO:22), and RNA10-1 (SEQ ID NO:23) to RNA10-2 (SEQ ID NO:24), respectively. Each dsRNA was transferred into 293 cells (ATCC No. CRL-1573) along with pGL3-3′ (GFP) and pRL-TK (Promega) as an internal control which expresses Renilla luciferase. Gene transfer was carried out using TransIT293 (Takara Bio) and TransIT-TKO (Takara Bio) . The siRNA concentrations were set to result in final concentrations of 1.25 to 160 nM. The cells were cultured for 24 hours. After the culture supernatant was removed, the cells were washed once with PBS, and lysed with a 5-fold dilution of 5×Passive Lysis Buffer (Promega) in deionized water. Values for luminescence from the firefly luciferase and the Renilla luciferase were measured for 10 μl of the lysate according to the protocol of Dual Luciferase Assay System (Promega) using a luminescence plate reader Mithras 940 (Berthold). Relative luminescence intensity (=value for luminescence from firefly luciferase/value for luminescence from Renilla luciferase) was calculated based on the thus obtained luminescence value. The results are shown in FIG. 3. In FIG. 3, the horizontal axis represents siRNA concentrations upon transfer, and the longitudinal axis represents relative values of relative luminescence intensities for the respective samples defining the relative luminescence intensity for control cells without the RNA transfer as 100%. As seen from FIG. 3, it was shown that lower relative values of relative luminescence intensities were observed for RNA8, RNA10, RNA9 and RNA7 in this order. Thus, it was judged that expression of the rsGFP gene is suppressed more effectively with the siRNAs, RNA8, RNA10, RNA9 and RNA7 in this order.

### Example 6

### Comparison of RNAi Effects Using Real-time RT-PCR

Each of the siRNAs (RNA7-10) and the rsGFP expression plasmid (pQBI25) were transferred into 293 cells using TransIT293 and TransIT-TKO. After two days, RNAs were extracted from the cells, and rsGFP mRNAs were quantified using real-time RT-PCR. Cells into which only pQBI25 was transferred were used as a control, and the data were corrected using the neomycin resistance gene in pQBI25. The real-time RT-PCR was carried out using Real Time RT-PCR Core Kit (Takara Bio) and Smart Cycler System (Takara Bio) as well as the GFP-B-F (SEQ ID NO:25) and GFP-B-R (SEQ ID NO:26) as primers for rsGFP or the oligo DNAs Neo-F (SEQ ID NO:27) and Neo-R (SEQ ID NO:28) as primers for the neomycin resistance gene. The results are shown in FIG. 4. In FIG. 4, the horizontal axis represents the names of siRNAs used, and the longitudinal axis represents relative values defining the mRNA amount for control cells into which only pQBI25 was transferred as 100. The decreases in rsGFP mRNA amounts as shown in FIG. 4 were consistent with the decreases in relative values of relative luminescence intensities from firefly luciferase observed in Example 5. Thus, it was confirmed that the siRNA screening method as described in Examples 4 and 5 is useful.

### INDUSTRIAL APPLICABILITY

The present invention provides a method of screening for a nucleotide molecule that suppresses expression of a target gene by destabilizing an RNA, which can be universally applied to many target genes.

Sequence Listing Free Text

SEQ ID NO:2: Chimeric oligonucleotide designed as RNA2-1. “nucleotides 1 to 19 are ribonucleotides-other nucleotides are deoxyribonucleotides”

SEQ ID NO:3: Chimeric oligonucleotide designed as RNA2-2. “nucleotides 1 to 19 are ribonucleotides-other nucleotides are deoxyribonucleotides”

SEQ ID NO:4: Chimeric oligonucleotide designed as RNA3-1. “nucleotides 1 to 19 are ribonucleotides-other nucleotides are deoxyribonucleotides”

SEQ ID NO:5: Chimeric oligonucleotide designed as RNA3-2. “nucleotides 1 to 19 are ribonucleotides-other nucleotides are deoxyribonucleotides”

SEQ ID NO:6: Chimeric oligonucleotide designed as RNA4-1. “nucleotides 1 to 19 are ribonucleotides-other nucleotides are deoxyribonucleotides”

SEQ ID NO:7: Chimeric oligonucleotide designed as RNA4-2. “nucleotides 1 to 19 are ribonucleotides-other nucleotides are deoxyribonucleotides”

SEQ ID NO:8: Chimeric oligonucleotide designed as RNA5-1. “nucleotides 1 to 19 are ribonucleotides-other nucleotides are deoxyribonucleotides”

SEQ ID NO:9: Chimeric oligonucleotide designed as RNA5-2. “nucleotides 1 to 19 are ribonucleotides-other nucleotides are deoxyribonucleotides”

SEQ ID NO:10: Chimeric oligonucleotide designed as RNA6-1. “nucleotides 1 to 19 are ribonucleotides-other nucleotides are deoxyribonucleotides”

SEQ ID NO:11: Chimeric oligonucleotide designed as RNA6-2. “nucleotides 1 to 19 are ribonucleotides-other nucleotides are deoxyribonucleotides”

SEQ ID NO:12: Designed PCR primer to amplify a portion of mouse Fas gene.

SEQ ID NO:13: Designed PCR primer to amplify a portion of mouse Fas gene.

SEQ ID NO:14: Designed PCR primer rsGFP-1 to amplify a portion of rsGFP gene.

SEQ ID NO:15: Designed PCR primer rsGFP-2 to amplify a portion of rsGFP gene.

SEQ ID NO:16: rsGFP gene

SEQ ID NO:17: Chimeric oligonucleotide designed as RNA7-1. “nucleotides 1 to 19 are ribonucleotides-other nucleotides are deoxyribonucleotides”

SEQ ID NO:18: Chimeric oligonucleotide designed as RNA7-2. “nucleotides 1 to 19 are ribonucleotides-other nucleotides are deoxyribonucleotides”

SEQ ID NO:19: Chimeric oligonucleotide designed as RNA8-1. “nucleotides 1 to 19 are ribonucleotides-other nucleotides are deoxyribonucleotides”

SEQ ID NO:20: Chimeric oligonucleotide designed as RNA8-2. “nucleotides 1 to 19 are ribonucleotides-other nucleotides are deoxyribonucleotides”

SEQ ID NO:21: Chimeric oligonucleotide designed as RNA9-1. “nucleotides 1 to 19 are ribonucleotides-other nucleotides are deoxyribonucleotides”

SEQ ID NO:22: Chimeric oligonucleotide designed as RNA9-2. “nucleotides 1 to 19 are ribonucleotides-other nucleotides are deoxyribonucleotides”

SEQ ID NO:23: Chimeric oligonucleotide designed as RNA10-1. “nucleotides 1 to 19 are ribonucleotides-other nucleotides are deoxyribonucleotides”

SEQ ID NO:24: Chimeric oligonucleotide designed as RNA10-2. “nucleotides 1 to 19 are ribonucleotides-other nucleotides are deoxyribonucleotides”

SEQ ID NO:25: Designed PCR primer GFP-B-F to amplify a portion of rsGFP gene.

SEQ ID NO:26: Designed PCR primer GFP-B-R to amplify a portion of rsGFP gene.

SEQ ID NO:27: Designed PCR primer Neo-F to amplify a portion of neomycin resistant gene.

SEQ ID NO:28: Designed PCR primer Neo-R to amplify a portion of neomycin resistant gene.

1. A nucleic acid construct having a promoter sequence, at least one protein-encoding nucleotide sequence linked to the promoter sequence in a translatable state, and a poly A signal sequence, wherein
the nucleic acid construct further contains, between the promoter sequence and the poly A signal sequence, a nontranslatable nucleotide sequence that is different from the protein-encoding nucleotide sequence,
the protein-encoding nucleotide sequence linked to the promoter sequence in a translatable state and the nontranslatable nucleotide sequence that is different from the protein-encoding nucleotide sequence are linked together so that they are transcribed from the nucleic acid construct in a single RNA molecule, and
the nontranslatable nucleotide sequence is selected from the group consisting of:
(1) a nucleotide sequence that encodes a protein or a part of the protein; and
(2) a nucleotide sequence of an untranslated region that is naturally located on the 5′ or 3′ side of a nucleotide sequence that encodes a protein.

2. The nucleic acid construct according to claim 1, wherein the nontranslatable nucleotide sequence is located downstream of the protein-encoding nucleotide sequence linked to the promoter sequence in a translatable state.

3. The nucleic acid construct according to claim 1, wherein the nontranslatable nucleotide sequence is located upstream of the protein-encoding nucleotide sequence linked to the promoter sequence in a translatable state.

4. The nucleic acid construct according to claim 1, the protein-encoding nucleotide sequence linked to the promoter sequence in a translatable state encodes a reporter protein.

5. A vector containing the nucleic acid construct defined by claim 1.

6. An RNA containing at least one protein-encoding nucleotide sequence in a translatable state and a nontranslatable nucleotide sequence that is different from the protein-encoding nucleotide sequence, wherein the nontranslatable nucleotide sequence is selected from the group consisting of:
(1) a nucleotide sequence that encodes a protein that is different from the protein-encoding nucleotide sequence in a translatable state, or a part of the protein; and
(2) a nucleotide sequence of an untranslated region that is naturally located on the 5′ or 3′ side of a nucleotide sequence that encodes a protein that is different from the protein-encoding nucleotide sequence in a translatable state.

7. A method of detecting an activity of altering expression of a target gene by a functional nucleotide molecule, the method comprising the steps of:
(1) transcribing an RNA from the nucleic acid construct defined by claim 1 or the vector defined by claim 5 which has, as a nontranslatable nucleotide sequence, a nucleotide sequence selected from the group consisting of a nucleotide sequence that encodes a protein in a target gene, a part of the nucleotide sequence, and an untranslated region that is located on the 5′ or 3′ side of the nucleotide sequence that encodes the protein in the target gene;
(2) contacting a nucleotide molecule with the RNA transcribed in step (1);
(3) detecting the RNA in step (2) or a translation product translated from the RNA; and
(4) detecting an activity of altering expression of the target gene by a functional nucleotide molecule based on the amount of the RNA or the translation product translated from the RNA detected in step (3).

8. A method of detecting an activity of altering expression of a target gene by a functional nucleotide molecule, the method comprising the steps of:
(1) contacting a nucleotide molecule with the RNA defined by claim 6 which has, as a nontranslatable nucleotide sequence, a nucleotide sequence selected from the group consisting of a nucleotide sequence that encodes a protein in a target gene, a part of the nucleotide sequence, and an untranslated region that is located on the 5′ or 3′ side of the nucleotide sequence that encodes the protein in the target gene;
(2) detecting the RNA in step (1) or a translation product translated from the RNA; and
(3) detecting an activity of altering expression of the target gene by a functional nucleotide molecule based on the amount of the RNA or the translation product translated from the RNA detected in step (2).

9. A method of screening for a functional nucleotide molecule that alters expression of a target gene, the method comprising detecting an activity of altering expression of a target gene by a functional nucleotide molecule according to the method defined by claim 7.

10. The method of detecting an activity of altering expression of a target gene by a functional nucleotide molecule according to claim 7, wherein the nucleotide molecule is contacted with the RNA in a cell or in a cell-free protein synthesis system.

11. A method of screening for a gene whose expression is altered by a nucleotide molecule, the method comprising the steps of:
(1) transcribing an RNA from the nucleic acid construct defined by claim 1 or the vector defined by claim 8 which has, as a nontranslatable nucleotide sequence, a nucleotide sequence selected from the group consisting of a nucleotide sequence that encodes a protein in an arbitrary gene, a part of the nucleotide sequence, and an untranslated region that is located on the 5′ or 3′ side of the nucleotide sequence that encodes the protein;
(2) contacting a nucleotide molecule with the RNA transcribed in step (1);
(3) detecting the RNA in step (2) or a translation product translated from the RNA; and
(4) identifying a gene whose expression is altered by the nucleotide molecule based on the amount of the RNA or the translation product translated from the RNA detected in step (3).

12. A method of screening for a gene whose expression is altered by a nucleotide molecule, the method comprising the steps of:
(1) contacting a nucleotide molecule with the RNA defined by claim 6 which has, as a nontranslatable nucleotide sequence, a nucleotide sequence selected from the group consisting of a nucleotide sequence that encodes a protein in an arbitrary gene, a part of the nucleotide sequence, and an untranslated region that is located on the 5′ or 3′ side of the nucleotide sequence that encodes the protein;
(2) detecting the RNA in step (1) or a translation product translated from the RNA; and
(3) identifying a functional nucleotide molecule that alters expression of a target gene based on the amount of the RNA or the translation product translated from the RNA detected in step (2).

13. The method of screening for a gene whose expression is altered by a nucleotide molecule according to claim 11, wherein the nucleotide molecule is contacted with the RNA in a cell or in a cell-free protein synthesis system.

14. A method of screening for a functional nucleotide molecule that alters expression of a target gene, the method comprising detecting an activity of altering expression of a target gene by a functional nucleotide molecule according to the method defined by claim 8.

15. The method of detecting an activity of altering expression of a target gene by a functional nucleotide molecule according to claim 8, wherein the nucleotide molecule is contacted with the RNA in a cell or in a cell-free protein synthesis system.

16. The method of screening for a gene whose expression is altered by a nucleotide molecule according to claim 12, wherein the nucleotide molecule is contacted with the RNA in a cell or in a cell-free protein synthesis system.
